(19) United States
(12) Patent Application Publication
(10) Pub. No.: US 2026/0103717 A1
(43) Pub. Date: Apr. 16, 2026

(54) PROMOTER FOR YEAST

(71) Applicant: BASF SE, Ludwigshafen am Rhein (DE)

(72) Inventors: Xuqiu Tan, San Diego, CA (US); Ruorong Cai, San Diego, CA (US); Jingping Zhong, San Diego, CA (US); Melissa Ann Scranton, San Diego, CA (US); Michael Speer, San Diego, CA (US)

(73) Assignee: BASF SE, Ludwigshafen am Rhein (DE)

(21) Appl. No.: 19/418,443

(22) Filed: Dec. 12, 2025

Related U.S. Application Data
(62) Division of application No. 18/512,485, filed on Nov. 17, 2023, now Pat. No. 12,522,838.
Application No. 18/512,485 is a division of application No. 15/734,682, now Pat. No. 11,866,714.
(60) Provisional application No. 62/682,053, filed on Jun. 7, 2018.

(51) Int. Cl. C12N 15/81; B29C 48/00; B29C 48/08; B29C 65/02; B29L 31/00; B65B 9/02; C07K 14/39; C08J 5/18; C08L 23/0807

(52) U.S. Cl. CPC C12N 15/815; B29C 48/022; B29C 48/08; B29C 65/02; B65B 9/023; C07K 14/39; C08J 5/18; C08L 23/0815; B29K 2023/06; B29L 2031/7128; C08J 2323/06; C08J 2323/08; C08J 2423/08

(57) ABSTRACT

A promoter operably linked to a gene encoding a protein is disclosed. The promoter drives expression of the protein in a yeast cell in the absence of methanol. Also disclosed are vectors, host cells and expression systems that include the promoter, as well as methods of using the promoter to express proteins in yeast.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### SEQUENCE LISTING

This application includes a nucleotide and amino acid sequence listing in computer readable form (CRF) as an XML file. The sequence listing is identified below and is hereby incorporated by reference into the specification of this application in its entirety and for all purposes.

| File Name | Date of Creation | Size (bytes) |
|---|---|---|
| BAF-23-1138US-DIV_SL.xml | Nov. 16, 2023 | 19.8 KB (20,336 bytes) |

### BACKGROUND

### Field

Despite numerous disadvantages to using methanol in protein expression, methanol inducible promoters, such as the AOX1 promoter (P_{AOX}1), have been widely used in Komagataella yeast expression systems for protein expression. As described herein, a promoter that can drive protein expression independently of methanol has been identified that works well with a variety of proteins for expression, such as enzymes.

### Description of the Related Art

Komagataella phaffii is a successful system for the production of a wide variety of recombinant proteins that are not native to the Komagataella cell. Several factors have contributed to its success as a protein manufacturing system, some of which include: (1) a promoter derived from the alcohol oxidase I (AOX1) gene of K. phaffii that is well suited for controlled expression of foreign genes; (2) similarity of techniques needed for the molecular genetic manipulation of K. phaffii to those of S. cerevisiae, which are well established; (3) the strong preference of K. phaffii for respiratory growth, which is a key physiological trait that facilitates its culturing at high-cell densities relative to fermentative yeasts; and (4) the knowledge base on the Komagataella system as described in numerous recent publications. Furthermore, the genome of several K. phaffii species have been sequenced, which allows facilitated studies of the RNA and protein expression pathways. The culturing condition of K. phaffii is also relatively easy, as the cells can grow in a high density culture with high levels of proteins being expressed at the intra- and extra-cellular level.

K. phaffii is a single-celled microorganism that is easy to manipulate and culture. K. phaffii is a eukaryote capable of many of the post-translational modifications performed by higher eukaryotic cells such as proteolytic processing, folding, disulfide bond formation and glycosylation. Thus, the system may help to avoid loss of proteins that may end up as inactive inclusion bodies in bacterial systems, as bacterial systems lack methods of post-translation modifications. Foreign proteins requiring post-translational modification may be produced as biologically active molecules in K. phaffii. Additionally, the K. phaffii system has been shown to give higher expression levels of protein than many bacterial systems.

The ability of K. phaffii to utilize methanol as a sole source of carbon and energy was discovered in the 1970s. There are two alcohol oxidase genes AOX1 and AOX2 which have strongly inducible promoters, the AOX promoters. These genes allow Komagataella to use methanol as a carbon and energy source. For example, the AOX1 protein is produced in response to depletion of some carbon sources, such as glucose, and the presence of methanol. In some cases, the gene encoding a desired heterologous protein can be introduced under the control of the AOX1 promoter, which means that gene expression and subsequent protein expression may be induced by the addition of methanol. As methanol could be synthesized from natural gas, methane, there was an interest in using these organisms for generating yeast biomass or single cell protein (SCP) to be marketed primarily as a high protein animal feed. During the 1970s, media and methods for growing K. phaffii on methanol in continuous culture at high cell densities (<130 g/l dry cell weight) were developed. However, during this same period, the cost of methane increased dramatically due to the oil crisis. Thus, the SCP process was never economically competitive for protein production.

Methods were then developed in the 1980's to produce K. phaffii as a heterologous gene expression system. The AOX1 gene (and its promoter) was isolated and vectors, strains and methods for molecular genetic manipulation of K. phaffii were developed. The combination of strong regulated expression under control of the AOX1 promoter along with the fermentation media and methods developed for the SCP process resulted in high levels of foreign proteins in K. phaffii.

Recombinant protein expression in K. phaffii may be driven by the promoter AOX1 and induced by methanol and repressed by other carbon sources such as glucose, glycerol and ethanol. This induction and repression feature functions as a switch which turns recombinant protein expression on and off under different culture conditions. This switch is advantageous when expressing proteins that are toxic towards the host cell and towards cell growth. However, there are several limitations with this system. As the AOX1 system requires methanol, the toxic and flammable material may require special handling and protocols. Additionally, hydrogen peroxide (H₂O₂) may be produced from methanol metabolism, which may also result in the degradation of recombinant proteins by the produced free radicals. The nature of methanol induction also limits where the manufacture location may be, and in some circumstances, may require long fermentation times and high biomass production. The production cost is considered to be high for a traditional Komagataella system (methanol inducible system).

As such, promoters that drive protein expression independently of methanol that work as well as or better than methanol inducible promoters, are sought. A promoter that may drive protein expression independently of methanol in yeast may reduce the protein expression cost and fermentation time. Additionally, there would be no need for food grade methanol in the process, thus allowing an easy and robust fermentation method for products such as edible and medical products. Thus, a promoter system for production of protein without the presence of methanol, or a constitutive promoter system would be advantageous for the expression of recombinant proteins in the K. phaffii system.

### SUMMARY

In a first aspect, a promoter comprising a nucleic acid sequence having at least of 55%, 60%, 65%, 70%, 75%, 80%, 85%, 90%, 99% or more sequence identity to any one of SEQ ID NO: 1-7, or a fragment thereof, wherein the promoter is operably linked to a gene encoding a protein, and the promoter drives the expression of the protein from a yeast cell in absence of methanol, is provided. In some embodiments, the sequence identity is over a region of at least 50, 75, 100, 150, 200, 250, 300, 350, 400, 450, 500, 550, 600, 650, 700, 750, 800, 850, 900, 950, 1000, 2050, 1100, 1150, or more residues, or the full length of the nucleic acid. In some embodiments, the fragment of Seq. ID No: 1-7 is over a region of at least 50, 75, 100, 150, 200, 250, 300, 350, 400, 450, 500, 550, 600, 650, 700, 750, 800, 850, 900, 950, 1000, 2050, 1100, 1150, or more residues, or the full length of the nucleic acid. In some embodiments, the protein is an enzyme, a peptide, an antibody, or a recombinant protein. In some embodiments, the enzyme is a lipase, amylase, xylanase, protease, glucoamylase, glucanase, mannanase, phytase, or cellulase. In some embodiments, the protein is glycosylated. In some embodiments, the protein comprises disulfide bonds. In some embodiments, the nucleic acid sequence is 50, 100, 200, 300, 400, 500, 600, 700, 800, 900, 1000, 1500, 2000, 3000, 4000 or 5000 bases upstream from a translational start site of the at least one sequence encoding the protein or any number of bases in between a range defined by any two aforementioned values upstream from the start site of the at least one sequence encoding the protein. In some embodiments, the yeast cell is a species of methylotrophic yeast. In some embodiments, the yeast cell is of the genus Komagataella. In some embodiments, the yeast cell is selected from: K. farinosa, K. anomala, K. heedii, K. guilliermondii, K. kluyveri, K. membranifaciens, K. norvegensis, K. ohmeri, K. pastoris, K. phaffii, K. methanolica and K. subpelliclosa. In some embodiments, the expression of protein is up to 40 g/l.

In a second aspect, a vector comprising the promoter of any one of the embodiments herein is provided. In some embodiments, the vector is a yeast integrative plasmid, episomal plasmid, centromere plasmid or artificial chromosome. In some embodiments, the vector comprises a selectable marker.

In a third aspect, a yeast cell comprising the promoter or the vector of any one of the embodiments herein is provided.

In a fourth aspect, a protein expression system comprising the yeast cell of any one of the embodiments herein is provided.

In a fifth aspect, a method of expressing protein in a yeast cell is provided. The method comprises providing a yeast cell, introducing the promoter or the vector of any one of any one of the embodiments herein into the cell, fermenting the yeast cell under at least one fermentation condition in the absence of methanol in a nutrient broth, harvesting the cells and recovering protein from the cells. In some embodiments, the protein is excreted or is intracellular. In some embodiments, the protein is an enzyme, a peptide, an antibody, or a recombinant protein. In some embodiments, the enzyme is lipase, amylase, xylanase, protease, glucosamylase, glucanase, mannanase, phytase, or cellulase. In some embodiments, the method further comprises driving protein expression. In some embodiments, the yeast cells are a species of methylotrophic yeast. In some embodiments, the yeast cells are of the genus Komagataella. In some embodiments, the yeast cells are selected from the group consisting of K. farinosa, K. anomala, K. heedii, K. guilliermondii, K. kluyveri, K. membranifaciens, K. norvegensis, K. ohmeri, K. pastoris, K. methanolic, K. phafii and K. subpelliclosa. In some embodiments, the yeast cell is K. phafii. In some embodiments, the nutrient broth comprises at least one carbon source. In some embodiments, the at least one carbon source is selected from a group consisting of dextrose, maltose, glucose, dextrin, glycerol, sorbitol, mannitol, lactic acid, acetate, xylose, or other partially hydrolyzed starches, and any mixtures thereof. In some embodiments, the concentrations of the at least one carbon source varies from 0.0 g/1, 0.5 g/L, 1 g/L, 2 g/L, 4 g/L, 6 g/L, 8 g/L, 10 g/L, 11 g/L, 12 g/L, 13 g/L, 14 g/L, 15 g/L, 16 g/L, 18 g/L, 20 g/L, 22 g/L, 24 g/L, 26 g/L, 28 g/L, 30 g/L, 35 g/L, 40 g/L, 45 g/L, 50 g/L, 55 g/L, or 60 g/L any concentration within a range defined by any two aforementioned values. In some embodiments, the method further comprises addition of the at least one carbon source by pulse or continuous feeding.

### BRIEF DESCRIPTION OF THE DRAWINGS

FIG. 1 demonstrates the expression of lipase under the control of the promoter pAOX1 or pSD001 in microtiter plates. Shown are protein PAGE gels in which expression of the lipase is shown under the control of pAOX1 or pSD001.

FIG. 2 demonstrates the lipase fermentation yields from 45 to 150 hours in a broth that is deficient in methanol and a broth for methanol induction. As shown, the promoter pSD001 drives more lipase expression in the absence of methanol than the pAOX1 promoter in methanol induction conditions over the same period of time.

FIG. 3 shows a schematic of the promoter pSD001 as three functional forms, a 1.5 kb promoter, a 1 kb promoter and a 0.66 kb promoter for driving expression of lipase 1 (1.5 kb promoter (A)) and lipase 2 (1.5 kb promoter (B), 1 kb promoter (C) and 0.66 kb promoter (D)). As shown in the protein PAGE gel, all variations of the promoter were able to drive expression of the lipases.

FIGS. 4A, 4B, and 4C are an array of protein gel assays which demonstrate the expression of amylase 1 and 2 and a xylanase under the control of the promoter pSD001 in yeast cells in methanol-free expression conditions.

FIG. 5 is a panel of protein gel assays which demonstrate the expression of lipase 1 and 3 under the control of several promoters (pSD001, pSD002, pSD003, pSD004, pSD005, pSD007, pSD008) in yeast cells in methanol-free expression conditions in microtiter plates.

FIG. 6 shows whole broth fermentation yields of lipase 3 expressed in yeast cells under the control of the promoters: pSD003, pSD004 and pSD007. As shown, the pSD007 promoter led to the most expression of lipase between the promoters under two different methanol-free fermentation conditions at 120 hrs.

### DETAILED DESCRIPTION

In the description that follows, the terms should be given their plain and ordinary meaning when read in light of the specification. One of skill in the art would understand the terms as used in view of the whole specification.

As used herein, “a” or “an” may mean one or more than one.

“About” as used herein when referring to a measurable value is meant to encompass variations of +20% or +10%, more preferably +5%, even more preferably +1%, and still more preferably +0.1% from the specified value.

“Methylotrophic yeast,” as described herein, have its plain and ordinary meaning when read in light of the specification, and may include but is not limited to, for example, a limited number of yeast species that can use reduced one-carbon compounds such as methanol or methane, and multi-carbon compounds that contain no carbon bonds, such as dimethyl ether and dimethylamine. For example, these species can use methanol as the sole carbon and energy source for cell growth. Without being limiting, methylotrophs may include the Genus Methanoscacina, Methylococcus capsulatus, Hansenula polymorpha, Candida boidinii, Komagataella pastoris and Komagataella phaffii, for example. In the embodiments described herein, a promoter that drive protein expression independently of methanol is provided for protein expression in a methylotrophic yeast cell.

“Komagataella phaffii,” has its plain and ordinary meaning when read in light of the specification, and may include but is not limited to, for example, a species of methylotrophic yeast. “Pichia phaffii” may also refer to the colloquial name as it has officially been renamed Komagataella phaffii (for the GS115 strain used herein) or it may be also referred to as Komagataella pastoris, depending on which lineage it has.

Komagataella is widely used for protein expression using recombinant DNA techniques since its alcohol oxidase promoters were isolated and cloned. Hence it is used in biochemical and genetic research in academia and the biotechnical industry as it can express a wide range of diverse genes as compared to other microorganism such as Psesudomonas, Bacillus, and Aspergillus. Furthermore, the protein product is easier to purify and leads to a clean product. Komagataella is well suited for protein expression as it has a high growth rate and is able to grow on a simple, inexpensive medium. K. phaffii can grow in either shaker flasks or a fermenter, which makes it suitable for both small and large scale expression. K. phaffii has two alcohol oxidase genes AOX1 and AOX2, which have strongly inducible promoters. These genes allow Komagataella to use methanol as a carbon and energy source. The AOX promoters are induced by methanol and are repressed by glucose, for example. Often, the gene for a desired heterologous protein is introduced under the control of the AOX1 promoter, which means that protein expression can be induced by the addition of methanol. In a popular expression vector, the desired protein is produced as a fusion product to the secretion signal of the α-mating factor from Saccharomyces cerevisiae (baker's yeast). This causes the protein to be secreted into the growth medium, which greatly facilitates subsequent protein purification. Komagataella also has advantages over S. cerevisiae as well. Komagataella can easily be grown in cell suspension in reasonably strong methanol solutions that would kill most other micro-organisms, a system that is difficult to set up and maintain. As the protein yield from expression in a microbe is roughly equal to the product of the protein produced per cell and the number of cells, this makes Komagataella of great use when trying to produce large quantities of protein without expensive equipment. However, Komagataella may be unable to produce proteins for which the host may lack the proper chaperones. As such, Komagataella may be co-transformed with a nucleic acid or a gene that encodes a chaperone for proper protein folding.

“Chaperone protein,” “molecular chaperones,” or “chaperones” have their plain and ordinary meaning when read in light of the specification, and may include but is not limited to, for example, proteins that assist the covalent folding or unfolding and the assembly or disassembly of other macromolecular structures. Chaperones are present when the macromolecules perform their normal biological functions and have correctly completed the processes of folding and/or assembly. The chaperones are concerned primarily with protein folding. In some embodiments of the promoter, the promoter may drive protein expression independently of methanol. The protein may be a recombinant protein, such as for example, an enzyme. In some embodiments, a chaperone is expressed with the enzyme, wherein the chaperone assists in the folding of the enzyme. In some embodiments, expression of a chaperone leads to a functional enzyme. In some embodiments, the chaperone is expressed with a recombinant protein. In some embodiments, the promotor produces constitutively and is independent on the presence of methanol.

The budding yeast of strain K. phaffii, can grow on methanol and has been widely used for over 30 years for heterologous protein expression. For example, over 70 products including therapeutic biologicals (mostly) and industrial enzymes have been produced using the K. phaffii system. Protein from the system may be either secreted (>16 g/L) or produced for intracellular expression (>20 g/L). Most enzyme companies produce enzymes using a native host or homologous expression of the enzyme. However no native enzymes from Komagataella have been discovered for industrial use. Also appreciated by those skilled in the art, are methods for genome sequencing and molecular tools available for strain manipulation. Growth of the cells, fermentation and expression process are also well developed as the system has a long history of safe use and is regulatory friendly. Methods of growth of a typical culture for protein expression can be appreciated by those of skill in the art. In the embodiments provided herein, K. phaffii is used as an expression host for the expression of protein in a methanol-free environment.

“Nucleic acid” or “nucleic acid molecule” have their plain and ordinary meaning when read in light of the specification, and may include but is not limited to, for example, polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acids can be either single stranded or double stranded. In some embodiments, a nucleic acid sequence encoding a fusion protein or recombinant protein is provided, wherein the protein expression is driven by a promoter that drives protein expression independent of methanol. In some embodiments, the nucleic acid comprises a promoter that is not inducible by methanol. In some embodiments, a cell comprising the nucleotide for protein expression that is independent of methanol is provided.

“Coding for” or “encoding” are used herein, and have their plain and ordinary meaning when read in light of the specification, and may include but is not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. In some embodiments, a vector comprises a nucleic acid encoding a protein, wherein the nucleic acid encoding the protein is under the influence of a promoter that drives protein expression independently of methanol.

A “nucleic acid sequence coding for a polypeptide” has its plain and ordinary meaning when read in light of the specification, and may include but is not limited to, for example, all nucleotide sequences that are degenerate versions of each other and that code for the same amino acid sequence.

“Vector,” “expression vector” or “construct” have their plain and ordinary meaning when read in light of the specification, and may include but is not limited to, for example, a nucleic acid used to introduce heterologous nucleic acids into a cell that has regulatory elements to provide expression of the heterologous nucleic acids in the cell. The vector, as described herein, is a nucleic acid molecule encoding a gene that is expressed in a host-cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. Vectors include but are not limited to plasmid, minicircles, yeast, and viral genomes. Available commercial vectors are known to those of skill in the art. Commercial vectors are available from European Molecular Biology Laboratory and Atum, for example.

A “promoter that drives protein expression independently of methanol,” has its plain and ordinary meaning when read in light of the specification, and may include but is not limited to, for example, a promoter that may allow an increase in the expression of a specific gene in the absence of methanol.

“Constitutive promoter,” has its plain and ordinary meaning when read in light of the specification, and may include but is not limited to, for example, a promoter that is active in most circumstances in the cell. In some embodiments, the promoter drives heterologous protein expression independent of methanol, in yeast. In some embodiments, the yeast cells are a species of methylotrophic yeast. In some embodiments, the yeast cells are of the genus Komagataella. In some embodiments, the promoter is a constitutive promoter that may drives expression in the absence of methanol.

“Protein expression,” “protein expression,” have their plain and ordinary meaning when read in light of the specification, and may include but is not limited to, for example, the biotechnological process of generating a specific protein. It may be achieved by the manipulation of gene expression in an organism such that it expresses large amounts of a recombinant gene. Without being limiting, this may include the transcription of the recombinant DNA to messenger RNA (mRNA), the translation of mRNA into polypeptide chains, which are ultimately folded into functional proteins and may be targeted to specific subcellular or extracellular locations.

“Fusion proteins” or “chimeric proteins” have their plain and ordinary meaning when read in light of the specification, and may include but is not limited to, for example, proteins created through the joining of two or more genes that originally coded for separate proteins or portions of proteins. The fusion proteins can also be made up of specific protein domains from two or more separate proteins. Translation of this fusion gene can result in a single or multiple polypeptides with functional properties derived from each of the original proteins. Recombinant fusion proteins can be created artificially by recombinant DNA technology for use in biological research or therapeutics. Such methods for creating fusion proteins are known to those skilled in the art. Some fusion proteins combine whole peptides and therefore can contain all domains, especially functional domains, of the original proteins. However, other fusion proteins, especially those that are non-naturally occurring, combine only portions of coding sequences and therefore do not maintain the original functions of the parental genes that formed them. In some embodiments, promoters are provided that drive protein expression independently of methanol and are useful in driving protein expression in yeast. In some embodiments, the promoter is useful in driving expression of a fusion protein.

“Promoter” has its plain and ordinary meaning when read in light of the specification, and may include but is not limited to, for example, a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription may also be characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993); incorporated by reference in its entirety), cyclic AMP response elements (CREs), serum response elements (SREs; Treisman, Seminars in Cancer Biol. 1:47 (1990); incorporated by reference in its entirety), glucocorticoid response elements (GREs), and binding sites for other transcription factors, such as CRE/ATF (O'Reilly et al., J. Biol. Chem. 267:19938 (1992); incorporated by reference in its entirety), AP2 (Ye et al., J. Biol. Chem. 269:25728 (1994); incorporated by reference in its entirety), SP1, cAMP response element binding protein (CREB; Loeken, Gene Expr. 3:253 (1993); incorporated by reference in its entirety) and octamer factors (see, in general, Watson et al., eds., Molecular Biology of the Gene, 4th ed. (The Benjamin/Cummings Publishing Company, Inc. 1987; incorporated by reference in its entirety)), and Lemaigre and Rousseau, Biochem. J. 303:1 (1994); incorporated by reference in its entirety). A promoter may be constitutively active, repressible or inducible. If a promoter is an inducible promoter, then the rate of transcription initiation increases in response to an inducing agent. In contrast, the rate of transcription initiation is not regulated by an inducing agent if the promoter is a constitutive promoter. Repressible promoters are also known. In some embodiments, a gene delivery polynucleotide or vector is provided. In some embodiments, the gene delivery polynucleotide comprises a promoter sequence. The promoter can be specific for bacterial, mammalian or yeast expression, for example. In some embodiments, wherein a nucleic acid encoding a protein of interest is provided, the nucleic acid further comprises a promoter sequence. In some embodiments, the promoter is specific for expression in yeast. In some embodiments, the promoter is a conditional, inducible or a constitutive promoter. In some embodiments, the promoter is a promoter that is useful in driving protein expression independently of methanol, wherein the promoter drives protein expression in a methanol-free media. The promoters isolated herein may be inducible or constitutive and may drive protein expression in the absence of methanol.

“Conditional” or “inducible” have their plain and ordinary meaning when read in light of the specification, and may include but is not limited to, for example, a nucleic acid construct that includes a promoter that provides for gene expression in the presence of an inducer and does not substantially provide for gene expression in the absence of the inducer. In some embodiments, the promoter is an inducible promoter. In some embodiments, the promoter is an inducible promoter for yeast protein expression.

“Regulatory element” has its plain and ordinary meaning when read in light of the specification, and may include but is not limited to, for example, a regulatory sequence, which is any DNA sequence that is responsible for the regulation of gene expression, such as promoters and operators. The regulatory element can be a segment of a nucleic acid molecule, which is capable of increasing or decreasing the expression of specific genes within an organism. In some alternatives described herein, the gene is under a control of a regulatory element.

“Host cell” has its plain and ordinary meaning when read in light of the specification, and may include but is not limited to, for example, a cell that is introduced with a nucleic acid or vector that encodes a protein or gene of interest. In some embodiments, the host cell is an isolated cell. In the embodiments, described herein, the host cell is a yeast cell. In some embodiments, the cell is a methylotroph yeast cell. In some embodiments, the yeast cell is of Komagataella phaffii. In some embodiments, promoters that drive protein expression independently of methanol that are useful in driving protein expression in yeast is provided. In some embodiments, the promoter drives heterologous protein expression in yeast. In some embodiments, the yeast cells are of the genus Komagataella. In some embodiments, the isolated host cell is a yeast cell. In some embodiments, the isolated host cell is Komagataella phaffii.

The term “gene expression” refers to the biosynthesis of a gene product. For example, in the case of a gene encoding a structural protein, gene expression involves transcription of the gene into mRNA and translation of mRNA into the structural protein.

“Protein” has its plain and ordinary meaning when read in light of the specification, and may include but is not limited to, for example, a macromolecule comprising one or more polypeptide chains. A protein can also comprise non-peptide components, such as carbohydrate groups. Carbohydrates and other non-peptide substituents, such as post-translational modifications, can be added to a protein by the cell in which the protein is produced, and will vary with the type of cell. Proteins are defined herein in terms of their amino acid backbone structures; substituents such as carbohydrate groups are generally not specified, but can be present nonetheless. In some embodiments, a gene delivery polynucleotide or vector, is provided for expression protein, in a methanol independent method, in a Komagataella system. In some embodiments, the gene delivery polynucleotide or vector further comprises a sequence for at least one protein.

“Gene” has its plain and ordinary meaning when read in light of the specification, and may include but is not limited to, for example, the molecular unit of heredity of a living organism, describing some stretches of deoxyribonucleic acids (DNA) and ribonucleic acids (RNA) that code for a polypeptide or for an RNA chain that has a function in the organism, and can be a locatable region in the genome of an organism.

Genetic modification performed by transformation is described herein. “Transformation” refers to transferring genetic material, such as, for example, nucleic acids, PCT amplified nucleic acids, or synthetic DNA or RNA, to a cell. Common techniques employed for transferring genetic material may use viruses or viral vectors, electroporation, and/or chemical reagents to increase cell permeability. In some alternatives herein, the isolated host cell is transformed by electroporation. In some embodiments, the isolated host cell is transformed by exposure to alkali cations in the presence of a vector, plasmid or DNA.

Various transformation techniques have been developed and can be appreciated by one of skill in the art. Thus, gene transfer and expression methods are numerous but essentially function to introduce and express genetic material in yeast cells

“Sequence Identity”, “% sequence identity”, “% identity”, “% identical” or “sequence alignment” means a comparison of a first amino acid sequence to a second amino acid sequence, or a comparison of a first nucleic acid sequence to a second nucleic acid sequence and is calculated as a percentage based on the comparison. The result of this calculation can be described as “percent identical” or “percent ID.”

Generally, a sequence alignment can be used to calculate the sequence identity by one of two different approaches. In the first approach, both mismatches at a single position and gaps at a single position are counted as non-identical positions in final sequence identity calculation. In the second approach, mismatches at a single position are counted as non-identical positions in final sequence identity calculation; however, gaps at a single position are not counted (ignored) as non-identical positions in final sequence identity calculation. In other words, in the second approach gaps are ignored in final sequence identity calculation. The difference between these two approaches, i.e. counting gaps as non-identical positions vs ignoring gaps, at a single position can lead to variability in the sequence identity value between two sequences.

A sequence identity is determined by a program, which produces an alignment, and calculates identity counting both mismatches at a single position and gaps at a single position as non-identical positions in final sequence identity calculation. For example program Needle (EMBOS), which has implemented the algorithm of Needleman and Wunsch (Needleman and Wunsch, 1970, J. Mol. Biol. 48:443-453), and which calculates sequence identity per default settings by first producing an alignment between a first sequence and a second sequence, then counting the number of identical positions over the length of the alignment, then dividing the number of identical residues by the length of an alignment, then multiplying this number by 100 to generate the % sequence identity [% sequence identity=(# of Identical residues/length of alignment)×100)].

A sequence identity can be calculated from a pairwise alignment showing both sequences over the full length, so showing the first sequence and the second sequence in their full length (“Global sequence identity”). For example, program Needle (EMBOSS) produces such alignments; % sequence identity=(# of identical residues/length of alignment)×100)].

A sequence identity can be calculated from a pairwise alignment showing only a local region of the first sequence or the second sequence (“Local Identity”). For example, program Blast (NCBI) produces such alignments; % sequence identity=(# of Identical residues/length of alignment)×100)].

The sequence alignment is preferably generated by using the algorithm of Needleman and Wunsch (J. Mol. Biol. (1979) 48, p. 443-453). Preferably, the program “NEEDLE” (The European Molecular Biology Open Software Suite (EMBOSS)) is used with the programs default parameter (gap open=10.0, gap extend-0.5 and matrix=EBLOSUM62 for proteins and matrix=EDNAFULL for nucleotides). Then, a sequence identity can be calculated from the alignment showing both sequences over the full length, so showing the first sequence and the second sequence in their full length (“Global sequence identity”). For example: % sequence identity=(# of identical residues/length of alignment)×100)].

The variant nucleic acids are described by reference to a nucleic acid sequence which is at least n % identical to the nucleic acid sequence of the respective parent enzyme with “n” being an integer between 80 and 100. The variant nucleic acids include sequences that are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical when compared to the full-length sequence of the parent nucleic acid according to SEQ ID Nos. 1-7, wherein the variant is a promoter.

The variant nucleic acid comprises at least one modification compared to the parent nucleic acid. The variant nucleic acid of the present invention comprises at least one nucleotide substitution, nucleotide insertion and/or nucleotide deletion compared to the parent nucleic acid.

The yeast Komagataella phaffii has been widely used as a heterologous protein expression host. Strong inducible promoters derived from methanol utilization genes or constitutive glycolytic promoters are typically used to drive gene expression. Notably, genes involved in methanol utilization are not only repressed by the presence of glucose, but also by glycerol.

As described herein, novel promoters that drive protein expression independently of methanol to drive high heterologous expression in scale-relevant fermentation conditions in Komagataella phaffii are provided. Use of the promoters may lower the overall biomass and reduce cost of the expression of protein. Thus, the promoters described herein, drive protein expression independently of methanol and are helpful for allowing robust and efficient high throughput screening in Komagataella.

As described herein, the identified promoters may influence heterologous gene expression using fermentation conditions.

Some promoters for expression of genes in the absence of methanol have been previously described. For example, inducible promoters have previously been published for small molecule induction. Without being limiting, current promoters that induced independently of methanol include SUC2, P_{CUP1}, P_{GAL1} P_{ADH}, for example. However, the inducers for these specific promoters can be expensive. In addition, carbon-source dependent promoters have also been published. These can rely on relatively expensive carbon sources and can also be repressed by glucose, such as P_{ADH2}, GLK1, HXK2 and P1S1, for example. Likewise, constitutive promoters have also been described, such as the glyceraldehyde-3-phosphate dehydrogenase (GAP). (Weinhandl et al. 2014; included by reference in its entirety herein).

A problem with such known systems of promoters that drive protein expression independently of methanol for Komagataella is that these promoters have a weaker activity compared to the methanol-inducible AOX1 promoters. Previous studies have focused on strong promoters from shaker flask conditions, which might not correlate well to performance in scale-relevant or full-scale fermentation conditions. An ideal promoter would be strongly induced under scale-relevant fermentation conditions.

Thus, promoters that drive protein expression independently of methanol, are commercially desired to enable robust processes of protein expression, low-cost medium components, and lower levels of biomass.

Described herein are identified Komagataella native promoters that are capable of driving protein expression in a media that lacks methanol.

The recombinant expression system driven by methanol induction has several limitations. As the promoter P_{AOX1} requires methanol, the methods require special handling and may not be suitable in the expression of edible and medical products. Additionally, the use of methanol may lead to the by-product hydrogen peroxide (H₂O₂) from methanol metabolism which is known to lead to oxidative stress, which may lead to the degradation of the recombinant protein one is wishing to express.

### Example 1: Expression of Proteins Under the Control of the Promoters

Expression vectors are constructed with the promoter regions upstream of a gene for expression of a fusion protein or an enzyme, such as lipase. Vectors for protein expression may be constructed with the promoter placed immediately upstream of the translational start site of a gene encoding the protein. Thus, in some embodiments, these vectors can be used for transforming cells for protein expression in the absence of methanol. In some embodiments the cells are Komagataella cells.

Protein expression from the Komagataella cells may be assayed under fermentation conditions. It should be expected that the promoters described herein will drive protein expression independent of methanol (SEQ ID NO: 1-7).

### Example 2 Expression of Proteins Under the Control of a Promoter

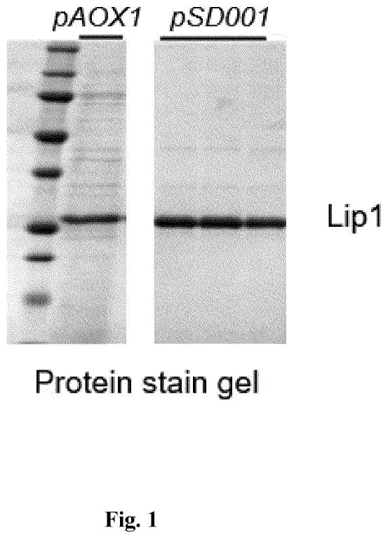

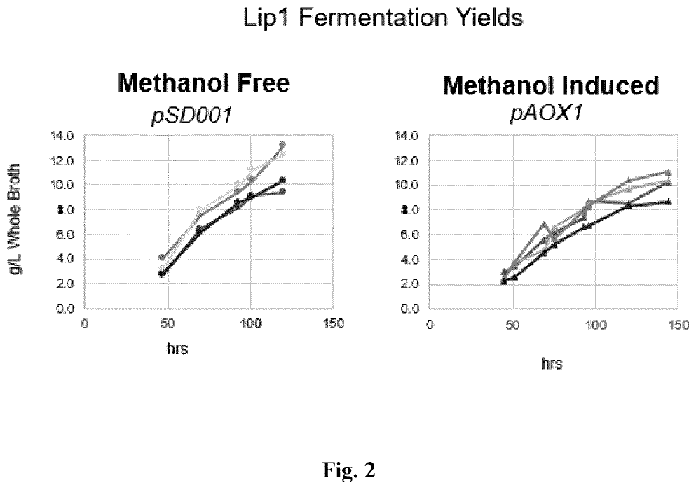

As shown in FIG. 1, the isolated promoter, pSD001 when compared to the control pAOX1, was able to drive expression of Lipase 1 in the absence of methanol in microtiter plates. Assays to measure the yield of lipase in fermentation broth were also performed which show that the pSD001 promoter led to expression of the marker protein, Lipase in fermentation broth in the absence of methanol (FIG. 2). In both microtiter plate and fermentation conditions, the expression of lipase 1 was higher in methanol-free conditions using the pSD001 promoter than in methanol induction conditions using the pAOX promoter.

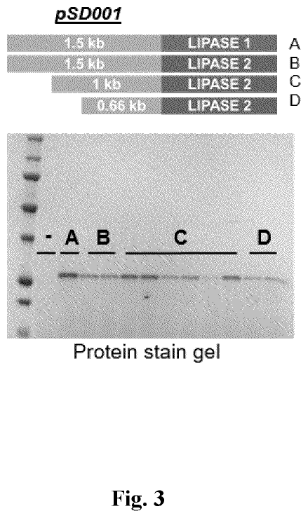

Several variations of the pSD001 promoter were constructed as shown in the diagram on FIG. 3. These constructs were then ligated to Lipase 1 or Lipase 2 genes and placed in an expression vector. As shown, a 1.5 kb promoter, a 1 kb promoter and a 0.66 kb promoter for driving expression of lipase 1 (1.5 kb promoter (A)) and lipase 2 (1.5 kb promoter (B), 1 kb promoter (C) and 0.66 kb promoter (D)) were used to drive lipase expression. As shown in FIG. 3, the protein PAGE gel, all variations of the promoters were able to drive expression of the lipases.

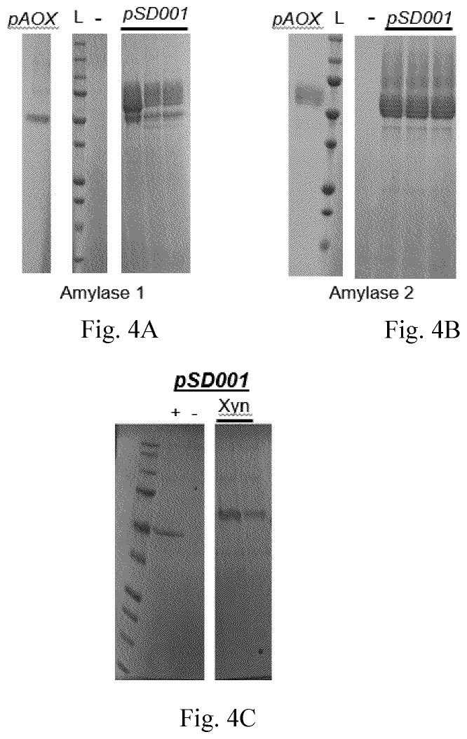

The pSD001 promoter was also tested for driving expression of other classes of enzymes. As shown in the panels, the promoter was able to drive expression of two amylases (amylase 1 and 2) and a xylanase in the absence of methanol. (FIG. 4A-4C).

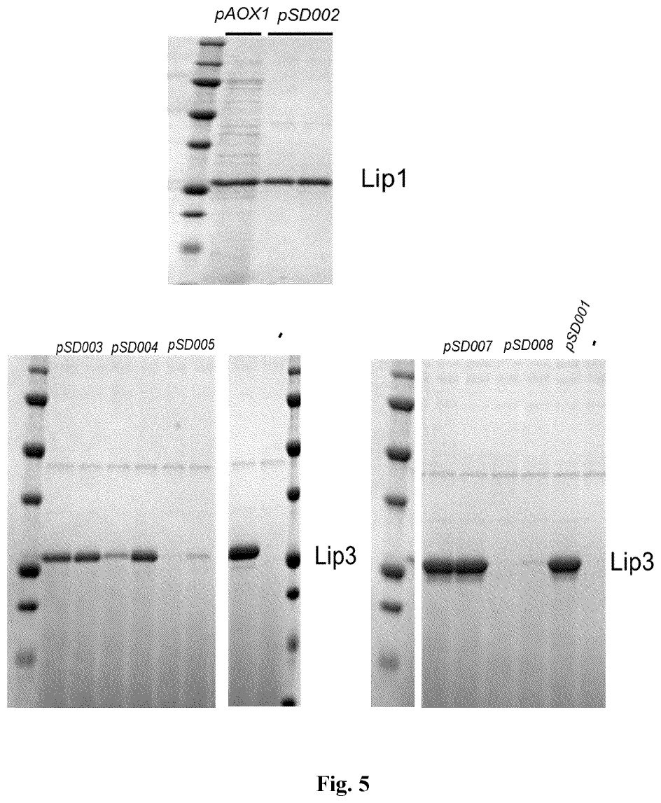

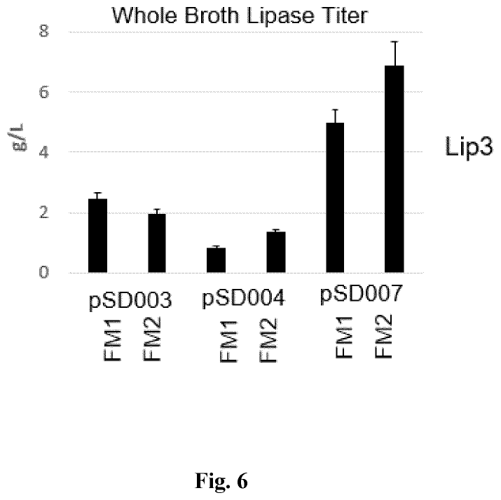

Various promoters were also tested for the ability to drive protein expression of lipase 1 and lipase 3 (Promoters: pSD001 (SEQ ID NO: 1), pSD002 (SEQ ID NO: 2), pSD003 (SEQ ID NO: 3), pSD004 (SEQ ID NO: 4), pSD005 (SEQ ID NO: 5), pSD007 (SEQ ID NO: 6) and pSD008 (SEQ ID NO: 7)). All promoters can drive lipase expression to various levels in microtiter plates, as shown in FIG. 5. The promoters: pSD003, SD004 and pSD007 were also tested in methanol-free fermentation conditions. As shown in FIG. 6, all three promoters led to lipase expression and the promoter pSD007 led to the most expression of protein.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of any one of the aspects is applicable to all aspects and embodiments identified herein. Moreover, any of the features of an embodiment any one of the aspects is independently combinable, partly or wholly with other embodiments described herein in any way, e.g., one, two, or three or more embodiments may be combinable in whole or in part. Further, any of the features of an embodiment of any one of the aspects may be made optional to other aspects or embodiments.

### Sequences

pSD001-
SEQ ID NO: 1
TCCAGTGTAGCACTAAAATCTAATATCTTCGGCTTTATACTTTTTTGTTC
ATCCGAAAGCTTACGAACAATTCTTTCTCCTGTTTTATTGTGGATATAGA
CAATTTCGTCAGTTTCTTGGAGAGAAGAGTTATTTCCGGTTTTGGCTGGC
CCTATAAACGGGTTCTTGGATTTGGATCTAGTAATAAAAATGTCACTGTC
ATTCTCGGAGCTGAACTTTGTGTTGTACGAAGATGGGTTGTTCCACTGTT
TTGCCAGCTCTTCATTGATGATTTTCTTAGTGGGTGTTCTTGGAGGTTCA
CGTTGCCTATAATCTTGACGTTCTTCTTCATCACTATCGATGCCATCAAA
ATTAAGCGTCCTTATTGCAGGCTTTTGTGATTTCAACTGCAATCCTTCTA
TCTCTTCATCAGAGCTTTCGAACTGAATACTATCACTCAAAACTGGCGAC
ATTGCACATTTCCGCAAACCATTTCGGGAATCTATGCTAGCTCTTCTAGA
CGATAAAGAACGACCGGAACCAATACGGGGTTGTGCAGGTGGGAATAAAT
ATGTTGGTTTGGATTCTTGACGTGAAGAAGGTATTCTAGTCGATGAAGTG
GTTGATAAGGATATGGCGTCACTGAGTTGTTTTCTTTTCCTATGTTGCGG
TGTTGGGTCAGGAGTTAATTGATTCACCTCCATAACTCTGGAATTTCTTG
AATGTGGGGTTTTCAGATGGGCATCTTTCTTGACGGGGTTGTGAGTAACG
GAGGAACCTGGTGTCTTGGGTGTGAACGGTGTTTGAGCCTGTACGCGGTT
ACTTCTGGGCGGAGTACTCGGAGTCATGAGAGCCATTGATTAGAAGGTGA
ATGAGGGAGTCACCACTCTAAGCAAACAAAATGAGGTCGAAGCAAAAAAT
AAAGTAAAGTAGCACTTCTGGCAGGTTAGATCAAAGAGTGACGGGAGATT
TGAAGATGGCTGGTTTTTCCTTAGTCTTGGAAGAGGTTTGTGTGGGTATC
AGCGAATATTCCCCGATTAGGCAAATTAGTTGCATTGAAATTAACACGAC
ATGGTGATTTGTGGTAACAAATATCTATTGGTGGTTGGTGTGTGGGTGTA
ATAGTGGTCGTGTCATGATGATGGTGTTCAGGTGTTGTCATAGATCGGTC
TTCAGTAAGAGAAGGAAGCTTGGTGACGATCACAGCTATGATGTAATAGA
AATTGCTAAGCAATTGTGAGGTGTGATGTATTTTGCAGAGCAATTGTGCG
GTACAACGGGGTGTTATTGTCTTCACAAGGCATTTATTGCGAATTTCGTA
GTTGAAAGAATATTTTAGCACAGGGTGCTTGACCCCTATTGTTGCTCGCT
AAACCATGATTGCTAAATGATGACATAGCAATCACTTTACTAAGATTGCT
ATAAGGACACCTTTCTTAGTATAAATGGACACTCTTTTCCCCTGCTAAAC
TTCTTTTATTTTTCACACTTAAACAGTTACAAAACACAAACACAACTAGA
A

pSD002-
SEQ ID NO: 2
GTGCTAAAATCTGAGGTTTACAAGCTGTGATGTTCCCCTAAGATCTCAC
AATCGAACAATCGCGAAGCCAATGCAAGTTGTTTAAGGGGAAACGACTC
ACTATTCCTGAAATTAGTATTCAAAACTTGGTCCGGAAGAACAATGAGG
CGGCCGTTAAAATACTCACGTAAACGGTGTCTACAAGCGCATTAAAATC
CGTTTGAATTCAAGCAAAAGCCACCAGAGGCTTATGCTTGGTTATACCC
AGCATTGACCTTTGGTATGAGCATCTGAAAAACAACCAGGTGTTGCAAA
GTTAAACATCCTTCTTTGTTCATATAGAACCCACTATTCATGGTACTCC
CCAATCGAATTTCACATTCTGGTTTTGAAATTACACACCACGTTAGCTT
ATAAGATTTCATATAACTTATTGATATACGGTTTCCATTGTTCGAATAG
TTGAGGTTGTATGTAATTCGATTGAAGGGGCCATTTTTGTTTCCTACTT
TTCCTGGGAGCTTATCCGATGCGCTTCAAAGCTGGAATTGTAAATATAG
AGAAAAAGAAGGATGTTGTTTTATTCTTGAAAGAGTATAATTTTACTTC
TAGCAACTCTCCCACTTCGCTTGACTTCATTTATTTCTTGGGCACATAG
GCGTAGTAATCTAGACCAACAGATAATTTGCCGGAATGATATAGCGATT
GGAAAATGAACTGAAATTTTTTGCTGTCTTTCAATTTGACGGGCAGTTC
ATCAGTGACCGACCATATAAATACGTTGAGAATGTTATTCTTCCTCGTA
GTTGAAGTGGCTTCATAATTTCAGAACTCAATAGATAAACTAGGATGTT
TTAAAGCAATTAATGCTCACAAGTAAGGAGCGACTCTCTTGCTTTTCGA
ATACTAAAAGTATCGTCCCAACCCAGAAAAAAAGACCTCTTAACTGCAA
AATAAACTCTATATATTTCTTCTAAAACAGTTTCAGGTTGGATAGTATC
GCATTCTCATCACTTCTAACTAGTAGGCCATGAGATATATTAACGTTTA
CTTGAGTTCTAAGTTCTCCGAATTAGATGCACAGCACAAACAAGATTAG
GTTTCACTTGGTACAAAATACGAACAGAGTTTAAGGTCGTAATTTCATT
TCGTTATTGATCCCCACAATCTATTCTTATCACAGTCATCAGATAGTCG
CGAAAAAGCATGCAGAAAAGGGGGTCGTCCCTATCTAAGTTGTAGCATT
ACAACAAATATGACTACACTCAGTGTCGCAATCGGTATAGCCAACGCTG
CAAAATGGATTCTACTGAGAATGGTATGATGATCCCAGGATCAATTTCC
CAAAAATTAAAAAAAGTAAAATAAAAAGCATCAGATATTAGGGAGGTGG
TAAGATTGCTCTGCAAGCGATCACGAGATTTTAGGTTTTCCTTTATGTA
CTATATAAAGCGCAGATTGGATGCCGCTTTTCCCTCCTGGGCTATGATA
ATATAGCGAACGAAATACACGCCAAAATAAA

pSD003-
SEQ ID NO: 3
TCACATTCATAGCATCTCTCGCCTGCAATAGCTTCCACGATAGGAATAT
CTGTGAAAGTGAACATGCTATTTCGATGATATAAGACTTTAAGATCTGG
CATGTTTGTGTTGGAGGTTACCCTGGGGTCAATAACCCTAATTATCTCC
TTCACTAAAAATGATGAAGATTCTTCGGATTCGTTTTTGAACAGAGTTA
ATGCCATTTCTTCGTCAATAGAAAAATCAATATCTGGTATCTCATCTTT
TACATATTGAGGATTTAGTTTTCTTCCCTTTGGATAGTACATTATGATC
AATGTATTCCTGTCTTTATTGATAAAGTATTGGCATTCTGCTTCTTGTA
CACCTTTGAATTGTTTGTCTGGAAGTGACTGACATTTTTCCACATTGCT
AACGGTTTGGCACGAATTACATCTAAATAAAATGTCTTCTCCGGATTCG
TGTATTAAGTGATACTCCAATGATAAATCCCCACCTATCGAACCAGAAT
CGGCATTGGCCACAGTCACAGGTAACTTTAGGTCTTGAAAAATCCTTCT
ATAGGCTTCATTGACATTGTCATAAGACTTAAGACCATCTTCTTTGGTC
AAGTCAAAAGAATAGGCATCTTTCATGAGAAACTCTCGTCCTCTCAACA
AACCTCCCCTAGGTCTCAACTCATCTCTATATTTGCGGGAAATTTGGTA
CACGAGAAGGGGTAAATCTTTATATGACGAACATAAGTCACCAACTAAG
TTTGTGATTTCCTCTTCACAAGTTGGCACTAAACAGTAGTCTCTATCCT
TGGAGTCTTTGAACTTGAACAATTCATTGTTGTCCCATCTCTTAGTTCT
CTCCCATAAATGCTTGGAAGACAGGCTACTTAATTCCATTTCCAGCCCA
CCAGCCTGATCCATTCTTTTCCTAATTACATTTTGAAGCTTTTTATAGG
TACGGAGTCCTAATGGAAGCCAGTGAACTATTCCTGCTGCAGGCTGGTA
AATAAACCTTGATTGAAGGAGCATATCATGAGTAGTAAGGTCCTTTACA
GAAAATAGTTTACTTCCTTGAAGAGAAGTAGAATAAAACCTCATGTTGG
GTCTCCATGAAAGGTTCAAAGGCATTGATCCTTTAGGTACTTCAGGATG
TTTAAGTCATCAAACTGTCCATCAAAGGTAGTATAGTATTTACCATCTA
GATAGTGATGTATGGGTGTAACACAACATTTAAATGTTGTAAATTAACA
TTAGGACTGAGTCCGGAGATGCTATTGTCACCTAAATCTATTAGAAAGC
ACTTCAGTTATATCATCGATAGAGGTTTGAAGATAAACCTATTGTTGAT
AAATAACCCCATTACCCGTTTACGTAGCAAGGTTCAAAAATTTGCTTAG
ATCGGAGCTAAAAATTCGACTGACTTCTTTCGAAAATGTGGATTATGCA
AGCAACGTTGCTATCGGAATAGTATATAAGGTCGATCTGCCCCATTACA
AATTGTAAAGCAACAAACATCCTACGCAAA

pSD004-
SEQ ID NO: 4
TCAGTTTCACGGTTATGTGAGCTGTCTCCGCGTGAGGCAGTAACCTCTG
TGTCATGGATACAGGCTGGTACACATTTGGCAGTAGGAACACAATCTGG
TTTAGTTGAAATATGGGACGCCACGACGTCCAAATGTACAAGATCAATG
ACTGGGCATTCGGCCCGAACCTCAGCGCTGAGTTGGAACCGTCATGTTT
TGAGTTCTGGTTCAAGAGATCGCAGTATCTTACATCGGGATGTACGTGC
AGCAGCTCACTATACAAGTCGCATTGTTGAACACCGCCAAGAGGTTTGT
GGCTTACGTTGGAACGTGGATGAAAACAAGCTGGCCAGTGGTTCCAATG
ATAACCGTATGATGGTATGGGATGCACTGCGTGTAGAACAGCCCCTTAT
GAAAGTTGAAGAGCATACTGCGGCTGTTAAGGCGTTGGCATGGTCACCT
CATCAACGTGGAATACTGGCTTCGGGTGGAGGTACTGCTGACAGACGTA
TCAAGGTGTGGAATACTTTAACAGGATCCAAGCTGCACGATGTTGATAC
TGGATCTCAAGTTTGTAATCTCTTGTGGTCTCGCAATTCTAATGAATTG
GTAAGTACTCATGGATATTCTCGAAACCAAGTCGTTATTTGGAAATATC
CGCAAATGAAGCAACTAGCATCTTTGACTGGTCATACTTATCGAGTCCT
TTACCTTTCCATGTCACCTGATGGAACTACAGTCGTAACGGGGGCTGGA
GACGAAACTTTAAGATTTTGGAACTGTTTCGAGAAGTCACGACAAAGCG
GAGGAGGATCAATATTACTAGACGCTTTTAGTCAGCTTCGTTAAATTAC
CACCAAATTTGGTGCAAAAGGGCCCATATGGTGCTACAACCAAAGGAAC
TTTCTAATTTTGATAATGATGTCATTTCTCTCATCGGGATGAAAATAGA
AGTCGAAAGGATTTTTGTCACTATTTCAAGCCCCACCTGCAGCTGGCAG
CATTTCTATTGTTTATGCATTGTCATTTATGGGAAAACTAAGAAAGTTC
CTCTCCACCCGGACTCCACTGGTAAATATGCGATATCGGAATCATGACC
AACCCATATTTTGATCCTAATCATTTCGGTTCTAGTCTCCGATCGGACT
CCGTAAAACTGCGGAGTGAACTCCAACGGAGAATACTGCAGCCAATCTC
ATATTTCATTTGTTATTTGTCCCTCAACTGTCTCGATAAGGTCATCTGT
GTTTGACTAGATGTTCGTCATTGGCATGTCAAACAAGGCTAGACCTTAC
AATCATCTCTTACGAATGTAAGTGAATGTAACTATATTTTCCTTGCTAC
TTTAACGAGGTTAACCAACCCCCGCACATCCCCACACCACCGCTCTTGA
TAAGCATCTCCGAAAATGCATGACGCGACAACTTCAAGCATGTTGTATT
TACTGAGTTTTCAGCCTCACTATCGATACCTCTATAAATAGAGGCACTT
TCGTCTCTTCTCCCTCCCCACAAGAAACCA

pSD005-
SEQ ID NO: 5
AGAAGTACTGTTATGAATCGATCGACGTGACATGTTGTTGATGGTTCTG
ACTTCTTGATGTCCGCGTTTTCTGTCTCTCAATAGTGGTGTTCGGGGGA
AGTATGGTTCTAATACTTAACAGGTAAGATGGTTGCAATGAGCACCTGG
TAAAGCAACTTGAATTTCCTGCCCTGTCTCCGTTAAGTTATATTCGACT
CAAGGTCCTTGCTTCCTGTCTGTTCTGTAAAACTTCCCTTTGGTGTCTT
CTATATCAACTTTAAAAACAAGGTAGTGTGTCGAGCGATAGTACTGTGT
CTTTTTCCCTATGAAAAAAATCGCACCATCCAAGACTTCTCACCTTCAA
CAGCTTCAACATCATGTTCGGTCCTTTTAGAGCTACGCTGGTCGATCTA
GGAGGTCTGCTATGGAAACGTCCTTGGAGAATGTCCAAACCACAGAAAT
ATAGACTCCGCAAAAGAATGCAACTTGTAGACTCCAATATCGACATTAT
TTACCAGGGACTGACTGAGGAGGGTCTGTCTTGCAAAGTGATAGATAAC
TTGAAACAAAACTTCCCAAAGGAGCATGAAGTGCTCCCCAAAAACAAGT
ATACCGTGTTTAACAAGACAGCCAAAAACTATAGAAAGGGTGTTCATTT
GGTTCCAAAATGGACCAAGAAGTCTTTGAGAGAGAACCCCGAGTTCTTC
TAATTGCACATTTCTTCCTGTTCATAGATTATCCCACACATAGTTGCTC
ACAAAAAAATCACTATAATTTTCCTCCACCGGCAGTATATCACTAACAC
CTTTATCTTTATTGTAGATTATAATCTGATCTTTATCCTTAGATGTATC
TATCATCAACCCCATGCTCTTGAAAAGCTTGAGTCTTAACACTGTCGAA
TCGTAGTTTTCTTGTAGATCATTCGATATCACTGCTTTTTCTTGCTCTT
CTAATTCGTTGAGATTCTGGGTCAAACTAGAGATTGAATTCTGAAGGTG
ATTCATGTTCATCTCCAGATCTGTTATTGATTTTGCTAATTTAAATTTT
TCGTGTTCAAGCTCTTCGATACTCTTTAGGGTCTGTTGACGGTCTTCTG
TTTCCAATAATTGCTTGTTGAACTCTTTAAGTTCGTCTCTCTGTTTACT
GATACGTGACAACAAATCTAGCTGGTGATCGAGTTTAAGTTTCCGTTTG
GAGCTCAACAGAGAAAGATTTTCATTAATTTGGTTGATAGTTTGCACGT
CCGGTTCGATCTGAAAATTCTCTATAGTCGACCTGATTAAGGACACAGT
CTCTTGAAGATCGGACATTGGATTTATGGAGAAGGGAGATCAAAGCGGA
ACCAGTTGCACTGTTTACCTTTCCAGTCGAGATACTTATCCCACAGGGC
CCTCACTTTCCAGGCAGAAGTCACCTAGGAGGCGCATCCCTCCGTTTGC
TTCCCTCGCGACAAACTCCCCTGTAAAAGAAAACTTCACTGAATCGTAC
ACCTAATCATACGACACTAACACAGATATA

pSD007-
SEQ ID NO: 6
GTCCTTTCCAAATTTTTGGTTGAAGGCATCGCTTAAATTATGAGCAGGA
TCGGTGGAAATAAGCAGGTATTTCTTGTTAGGATTGTGAAGGGCAAGCT
GGATAGATATAGAAGAAGATGTCGTGGTTTTACCGACACCCCCCTTACC
TCCAACAAAGATCCACTTCAGCGATTCGTGGTTCACAATTGATCGCAAA
CTTGGCTCTGCCTCAATATCCATGGTTGATGTCTAGTTGAGTGGCGTTT
GTGGTCTCTTGATGAGTTCAAGGCGAAAGAATATGATAGGAAAGCATGG
TTTGAACTTTTCGCGAAAGAAGGAATACTGTTCCGCGAGAAACTCCCCG
GTGCCAGAACCTTCCATTGAGGTTAATCGGTGGGAGGTGTTCGAATGAC
AATGTCAGACAAGGCGAACACGTCTTGTGACACCAGCTGGACTAAGAAG
ATTCGGTATGCACCGAAGAAGAAGGCCGTGTCTCAATTGGCAACTTTGC
AACAAACTACGGAGGAAAAGTCTCACAAGCTTTTAACCAAGTTGAATCA
CGACGACAACGATAAAGAAATCCTCAACCATCTAACACATGAAGTACAA
AGTAGAAATGTGATCTTATTGGACAAACTAGAGGAGCTCAACAAGGAAC
TGGGCTGGATTAAAGACCGAAAATGAGGAACCATGAGCACTGGGCGTTT
CCAGAAAAACTGCAACCAACGATGGGAAAATGATACCACACTACTATGG
TCACCCCACATTGTGAAATTTCAAACCAAAAAAGATCAACCCCATAATT
CCCCAGAGGGTTTTCCCAACAATTTTCCAACGGACTTGATAATGAGTCA
GATCATTTGAGCATATTCATCTTACCCCTTATTCCGTGACAATTTACCT
ATTCCATTCAAAGCATACGGTATCCCGTGACCTTCTCATGGAGATCATT
CTCCACCGATACAGCATATACACAGATATACCCAACTAATATCAATTGG
ACCTTGATATGGTCGACCTTGATGGTCCCGTCCAACCTTAAAACTTAGT
TTAATGCTATACTTTCGCCTTGAACCAAATCTGTCTCCCCCTCAATCAT
CTCTATGCAAGAAGGTCAACACTGATTACGTGAGCAACAGCCAGCAATC
GTTCGAGTCCCCGCCAAAAAAGGCGGAGTTACTGCTCCTTGTGACCACA
CCCCCTGAGACCACGTCCCTAAACGATCCTTGTCGGTTCCTTCGTCCAA
TTGGCAATTGCCACGCATACGTGAATCGTTATTGTTTCGCCTACCTTGC
GTCATTCGTTCCAGAATGTTCGACATACTCCTCTAGAACATACCGTCAC
ACCACCATCTTAAGTTATCTTCACGTGACCATGACGTACATTGTAGTTG
ACTACCCCATTCTCATCATTCCGATGCGGCCAAAAATCTCTATATAAAG
ACCGTATCCCCTAATATTCTCTTCTTGTTAAGACATTAACTTAGTTAAT
TCACCAATTACTCACTTATAAACAAACAAA

pSD008-
SEQ ID NO: 7
GTTTCTCTTGGGGAGATACTTTTTTCGCGTGCTCCTCCGTGCGGAACTT
CCTTCTGAGCTTCTACCTCTCAGATTAGTCTAATCGCATCAGGAATAAG
ACTGAGAATGCTTTTAAGGAGAGGCTTGAGATTGGCTAATTGCGTTCCG
AAGTACTCTTTCAAAAGGAGTTATACCCCTCTCAACTACGATTCTCTAA
AGAATTATCGTAGGCATGCTCAGGCGCCTCAACCCCATCAGTTTGACGC
CACTAGATGGGACCAACAACCAGTTACTAATGAGCAAGGAGTAATACTC
CCATCCGACTCAATTGCAAACATTCTGAGACAACCAACTCTGGTCATAG
AACGGCAAATGGAAATGATGAATATATTTTTAGGATTTGAGCAGGCGAA
CCGATATGTTATCATGGATCCTACAGGAAGTATTTTGGGTTACATGCTA
GAAAGGGATCTGGGCATCACCAAAGCTATATTGAGACAGATCTACCGTT
TGCATCGACCTTTTACAGTGGATGTAATGGATACTGCAGGAAATGTATT
AATGACAATCAAGAGGCCGTTTAGTTTCATCAATTCGCACATCAAAGCT
ATATTACCCCCTTTCAGGAACAGCGACCCAGACGAACATGTAATTGGAG
AATCCGTTCAAAGCTGGCATCCTTGGAGACGAAGATACAATCTATTTAC
AGCACAAATTGGCGAAAAGGACACTGTCTACGATCAGTTCGGGTACATT
GACGCACCGTTTCTTTCCTTTGAGTTTCCTGTACTTTCAGAATCTAGGC
AAACGCTAGGTGCTGTCTCTAGAAACTTCGTGGGCTTTGCAAGAGAGCT
TTTCACAGATACAGGAGTTTACATCATCCGTATGGGGCCTGAATCTTTT
GTAGGGCTAGAAGGGAACTACGGGAACAATGTGGCCCAACATGCCCTTA
CGCTGGACCAAAGGGCTGTATTATTAGCCAATGCCGTTTCAATTGACTT
TGATTACTTTTCTAGGCACTCGTCACACAGTGGTGGCTTCATTGGGTTT
GAGGAATAGACAGGGTCTCGTCAACTCAGCTCCTGCCACCAAACCAATC
ATTGATCAACGAGCACACTTTTGTCCACGTGAGATCGCTTTCGCTTGCA
GAAAGAGCAATGCATGAAAACGGCAAACGCAAAACGAGCAAAAAAACGA
GTAAATAACTACAATTTCACCACCAACAGGGTCAAAGAGCTTTTGAGAC
ACTATAAAAGGGGCCCTTTCCCCCCAGGTTCCTTGAAATCCTCATTCAA
TTATGTTTTTTACTCATAATTTGACTCAATTGGCATCTTCTTCTTTGTT
CATATACAGTAATTGATATGACGCTTAGTCATTATTAGTGTTCTCGACT
AGCAGTGGCGAAAAAAGGGGGAGTTATTTTCTAGAACCGACCGCAAACT
ATAAAAGAAAGCTGCCCCTCATATACCTTTCGAATTCTTTATTTTCTGT
GTTTCTTCCCTATTTAACATCTACACAAAA

What is claimed is:

1. An isolated nucleic acid sequence having at least of 55%, 60%, 65%, 70%, 75%, 80%, 85%, 90%, 99% or more sequence identity to any one of SEQ ID NO: 1-7, wherein the nucleic acid sequence is operably linked to a gene encoding a protein, and the nucleic acid sequence drives expression of the protein in a yeast cell in the absence of methanol, wherein the yeast cell is of the genus Komagataella, and the yeast cell is selected from: K. farinosa, K. anomala, K. heedii, K. guilliermondii, K. kluyveri, K. membranifaciens, K. norvegensis, K. ohmeri, K. pastoris, K. phaffii, K. methanolica and K. subpelliclosa.

2. The isolated nucleic acid sequence of claim 1, wherein the sequence identity is over a region of at least 50, 75, 100, 150, 200, 250, 300, 350, 400, 450, 500, 550, 600, 650, 700, 750, 800, 850, 900, 950, 1000, 2050, 1100, 1150, or more residues, or the full length of the nucleic acid.

3. The isolated nucleic acid sequence of claim 1, wherein the fragment is over a region of at least 50, 75, 100, 150, 200, 250, 300, 350, 400, 450, 500, 550, 600, 650, 700, 750, 800, 850, 900, 950, 1000, 2050, 1100, 1150, or more residues, or the full length of the nucleic acid.

4. The isolated nucleic acid sequence of claim 1, wherein the protein is an enzyme, a peptide, an antibody, or a recombinant protein.

5. The isolated nucleic acid sequence of claim 1, wherein the enzyme is a lipase, amylase, xylanase, protease, glucoamylase, glucanase, mannanase, phytase, or cellulase.

6. The isolated nucleic acid sequence of claim 1, wherein the protein is glycosylated or non-glycosylated.

7. The isolated nucleic acid sequence of claim 1, wherein the protein optionally comprises disulfide bonds.

8. The isolated nucleic acid sequence of claim 1, wherein the nucleic acid sequence is 50, 100, 200, 300, 400, 500, 600, 700, 800, 900, 1000, 1500, 2000, 3000, 4000 or 5000 bases upstream from a translational start site of the at least one sequence encoding the protein or any number of bases in between a range defined by any two aforementioned values upstream from the start site of the at least one sequence encoding the protein.

9. The isolated nucleic acid sequence of claim 1, wherein the yeast cell are a species of methylotropic yeast.

10. The isolated nucleic acid sequence of claim 1, wherein the expression of protein is up to 40 g/l.

11. A vector comprising the isolated nucleic acid sequence of claim 1.

12. The vector of claim 11, wherein the vector is a yeast integrative plasmid, episomal plasmid, centromere plasmid or artificial chromosome.

13. The vector of claim 12 wherein the vector comprises a selectable marker.

14. A yeast cell comprising the isolated nucleic acid sequence of claim 1.

15. A protein expression system comprising the yeast cell of claim 14.


SEQUENCE LISTING (7 sequences)

SEQ ID NO: 1 - DNA, 1501 bp, synthetic construct
  misc_feature 1..1501: note = Description of Artificial Sequence: Synthetic Oligonucleotide
  source 1..1501: mol_type = other DNA
tccagtgtagcactaaaatctaatatcttcggctttatacttttttgttcatccgaaagcttacgaacaattctttctcctgttttattgtggatatagacaatttcgtcagtttcttggagagaagagttatttccggttttggctggccctataaacgggttcttggatttggatctagtaataaaaatgtcactgtcattctcggagctgaactttgtgttgtacgaagatgggttgttccactgttttgccagctcttcattgatgattttcttagtgggtgttcttggaggttcacgttgcctataatcttgacgttcttcttcatcactatcgatgccatcaaaattaagcgtccttattgcaggcttttgtgatttcaactgcaatccttctatctcttcatcagagctttcgaactgaatactatcactcaaaactggcgacattgcacatttccgcaaaccatttcgggaatctatgctagctcttctagacgataaagaacgaccggaaccaatacggggttgtgcaggtgggaataaatatgttggtttggattcttgacgtgaagaaggtattctagtcgatgaagtggttgataaggatatggcgtcactgagttgttttcttttcctatgttgcggtgttgggtcaggagttaattgattcacctccataactctggaatttcttgaatgtggggttttcagatgggcatctttcttgacggggttgtgagtaacggaggaacctggtgtcttgggtgtgaacggtgtttgagcctgtacgcggttacttctgggcggagtactcggagtcatgagagccattgattagaaggtgaatgagggagtcaccactctaagcaaacaaaatgaggtcgaagcaaaaaataaagtaaagtagcacttctggcaggttagatcaaagagtgacgggagatttgaagatggctggtttttccttagtcttggaagaggtttgtgtgggtatcagcgaatattccccgattaggcaaattagttgcattgaaattaacacgacatggtgatttgtggtaacaaatatctattggtggttggtgtgtgggtgtaatagtggtcgtgtcatgatgatggtgttcaggtgttgtcatagatcggtcttcagtaagagaaggaagcttggtgacgatcacagctatgatgtaatagaaattgctaagcaattgtgaggtgtgatgtattttgcagagcaattgtgcggtacaacggggtgttattgtcttcacaaggcatttattgcgaatttcgtagttgaaagaatattttagcacagggtgcttgacccctattgttgctcgctaaaccatgattgctaaatgatgacatagcaatcactttactaagattgctataaggacacctttcttagtataaatggacactcttttcccctgctaaacttcttttatttttcacacttaaacagttacaaaacacaaacacaactagaa

SEQ ID NO: 2 - DNA, 1501 bp, synthetic construct
  misc_feature 1..1501: note = Description of Artificial Sequence: Synthetic Oligonucleotide
  source 1..1501: mol_type = other DNA
gtgctaaaatctgaggtttacaagctgtgatgttcccctaagatctcacaatcgaacaatcgcgaagccaatgcaagttgtttaaggggaaacgactcactattcctgaaattagtattcaaaacttggtccggaagaacaatgaggcggccgttaaaatactcacgtaaacggtgtctacaagcgcattaaaatccgtttgaattcaagcaaaagccaccagaggcttatgcttggttatacccagcattgacctttggtatgagcatctgaaaaacaaccaggtgttgcaaagttaaacatccttctttgttcatatagaacccactattcatggtactccccaatcgaatttcacattctggttttgaaattacacaccacgttagcttataagatttcatataacttattgatatacggtttccattgttcgaatagttgaggttgtatgtaattcgattgaaggggccatttttgtttcctacttttcctgggagcttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaa

SEQ ID NO: 3 - DNA, 1500 bp, synthetic construct
  misc_feature 1..1500: note = Description of Artificial Sequence: Synthetic Oligonucleotide
  source 1..1500: mol_type = other DNA
tcacattcatagcatctctcgcctgcaatagcttccacgataggaatatctgtgaaagtgaacatgctatttcgatgatataagactttaagatctggcatgtttgtgttggaggttaccctggggtcaataaccctaattatctccttcactaaaaatgatgaagattcttcggattcgtttttgaacagagttaatgccatttcttcgtcaatagaaaaatcaatatctggtatctcatcttttacatattgaggatttagttttcttccctttggatagtacattatgatcaatgtattcctgtctttattgataaagtattggcattctgcttcttgtacacctttgaattgtttgtctggaagtgactgacatttttccacattgctaacggtttggcacgaattacatctaaataaaatgtcttctccggattcgtgtattaagtgatactccaatgataaatccccacctatcgaaccagaatcggcattggccacagtcacaggtaactttaggtcttgaaaaatccttctataggcttcattgacattgtcataagacttaagaccatcttctttggtcaagtcaaaagaataggcatctttcatgagaaactctcgtcctctcaacaaacctcccctaggtctcaactcatctctatatttgcgggaaatttggtacacgagaaggggtaaatctttatatgacgaacataagtcaccaactaagtttgtgatttcctcttcacaagttggcactaaacagtagtctctatccttggagtctttgaacttgaacaattcattgttgtcccatctcttagttctctcccataaatgcttggaagacaggctacttaattccatttccagcccaccagcctgatccattcttttcctaattacattttgaagctttttataggtacggagtcctaatggaagccagtgaactattcctgctgcaggctggtaaataaaccttgattgaaggagcatatcatgagtagtaaggtcctttacagaaaatagtttacttccttgaagagaagtagaataaaacctcatgttgggtctccatgaaaggttcaaaggcattgatcctttaggtacttcaggatgtttaagtcatcaaactgtccatcaaaggtagtatagtatttaccatctagatagtgatgtatgggtgtaacacaacatttaaatgttgtaaattaacattaggactgagtccggagatgctattgtcacctaaatctattagaaagcacttcagttatatcatcgatagaggtttgaagataaacctattgttgataaataaccccattacccgtttacgtagcaaggttcaaaaatttgcttagatcggagctaaaaattcgactgacttctttcgaaaatgtggattatgcaagcaacgttgctatcggaatagtatataaggtcgatctgccccattacaaattgtaaagcaacaaacatcctacgcaaa

SEQ ID NO: 4 - DNA, 1500 bp, synthetic construct
  misc_feature 1..1500: note = Description of Artificial Sequence: Synthetic Oligonucleotide
  source 1..1500: mol_type = other DNA
tcagtttcacggttatgtgagctgtctccgcgtgaggcagtaacctctgtgtcatggatacaggctggtacacatttggcagtaggaacacaatctggtttagttgaaatatgggacgccacgacgtccaaatgtacaagatcaatgactgggcattcggcccgaacctcagcgctgagttggaaccgtcatgttttgagttctggttcaagagatcgcagtatcttacatcgggatgtacgtgcagcagctcactatacaagtcgcattgttgaacaccgccaagaggtttgtggcttacgttggaacgtggatgaaaacaagctggccagtggttccaatgataaccgtatgatggtatgggatgcactgcgtgtagaacagccccttatgaaagttgaagagcatactgcggctgttaaggcgttggcatggtcacctcatcaacgtggaatactggcttcgggtggaggtactgctgacagacgtatcaaggtgtggaatactttaacaggatccaagctgcacgatgttgatactggatctcaagtttgtaatctcttgtggtctcgcaattctaatgaattggtaagtactcatggatattctcgaaaccaagtcgttatttggaaatatccgcaaatgaagcaactagcatctttgactggtcatacttatcgagtcctttacctttccatgtcacctgatggaactacagtcgtaacgggggctggagacgaaactttaagattttggaactgtttcgagaagtcacgacaaagcggaggaggatcaatattactagacgcttttagtcagcttcgttaaattaccaccaaatttggtgcaaaagggcccatatggtgctacaaccaaaggaactttctaattttgataatgatgtcatttctctcatcgggatgaaaatagaagtcgaaaggatttttgtcactatttcaagccccacctgcagctggcagcatttctattgtttatgcattgtcatttatgggaaaactaagaaagttcctctccacccggactccactggtaaatatgcgatatcggaatcatgaccaacccatattttgatcctaatcatttcggttctagtctccgatcggactccgtaaaactgcggagtgaactccaacggagaatactgcagccaatctcatatttcatttgttatttgtccctcaactgtctcgataaggtcatctgtgtttgactagatgttcgtcattggcatgtcaaacaaggctagaccttacaatcatctcttacgaatgtaagtgaatgtaactatattttccttgctactttaacgaggttaaccaacccccgcacatccccacaccaccgctcttgataagcatctccgaaaatgcatgacgcgacaacttcaagcatgttgtatttactgagttttcagcctcactatcgatacctctataaatagaggcactttcgtctcttctccctccccacaagaaacca

SEQ ID NO: 5 - DNA, 1500 bp, synthetic construct
  misc_feature 1..1500: note = Description of Artificial Sequence: Synthetic Oligonucleotide
  source 1..1500: mol_type = other DNA
agaagtactgttatgaatcgatcgacgtgacatgttgttgatggttctgacttcttgatgtccgcgttttctgtctctcaatagtggtgttcgggggaagtatggttctaatacttaacaggtaagatggttgcaatgagcacctggtaaagcaacttgaatttcctgccctgtctccgttaagttatattcgactcaaggtccttgcttcctgtctgttctgtaaaacttccctttggtgtcttctatatcaactttaaaaacaaggtagtgtgtcgagcgatagtactgtgtctttttccctatgaaaaaaatcgcaccatccaagacttctcaccttcaacagcttcaacatcatgttcggtccttttagagctacgctggtcgatctaggaggtctgctatggaaacgtccttggagaatgtccaaaccacagaaatatagactccgcaaaagaatgcaacttgtagactccaatatcgacattatttaccagggactgactgaggagggtctgtcttgcaaagtgatagataacttgaaacaaaacttcccaaaggagcatgaagtgctccccaaaaacaagtataccgtgtttaacaagacagccaaaaactatagaaagggtgttcatttggttccaaaatggaccaagaagtctttgagagagaaccccgagttcttctaattgcacatttcttcctgttcatagattatcccacacatagttgctcacaaaaaaatcactataattttcctccaccggcagtatatcactaacacctttatctttattgtagattataatctgatctttatccttagatgtatctatcatcaaccccatgctcttgaaaagcttgagtcttaacactgtcgaatcgtagttttcttgtagatcattcgatatcactgctttttcttgctcttctaattcgttgagattctgggtcaaactagagattgaattctgaaggtgattcatgttcatctccagatctgttattgattttgctaatttaaatttttcgtgttcaagctcttcgatactctttagggtctgttgacggtcttctgtttccaataattgcttgttgaactctttaagttcgtctctctgtttactgatacgtgacaacaaatctagctggtgatcgagtttaagtttccgtttggagctcaacagagaaagattttcattaatttggttgatagtttgcacgtccggttcgatctgaaaattctctatagtcgacctgattaaggacacagtctcttgaagatcggacattggatttatggagaagggagatcaaagcggaaccagttgcactgtttacctttccagtcgagatacttatcccacagggccctcactttccaggcagaagtcacctaggaggcgcatccctccgtttgcttccctcgcgacaaactcccctgtaaaagaaaacttcactgaatcgtacacctaatcatacgacactaacacagatata

SEQ ID NO: 6 - DNA, 1500 bp, synthetic construct
  misc_feature 1..1500: note = Description of Artificial Sequence: Synthetic Oligonucleotide
  source 1..1500: mol_type = other DNA
gtcctttccaaatttttggttgaaggcatcgcttaaattatgagcaggatcggtggaaataagcaggtatttcttgttaggattgtgaagggcaagctggatagatatagaagaagatgtcgtggttttaccgacaccccccttacctccaacaaagatccacttcagcgattcgtggttcacaattgatcgcaaacttggctctgcctcaatatccatggttgatgtctagttgagtggcgtttgtggtctcttgatgagttcaaggcgaaagaatatgataggaaagcatggtttgaacttttcgcgaaagaaggaatactgttccgcgagaaactccccggtgccagaaccttccattgaggttaatcggtgggaggtgttcgaatgacaatgtcagacaaggcgaacacgtcttgtgacaccagctggactaagaagattcggtatgcaccgaagaagaaggccgtgtctcaattggcaactttgcaacaaactacggaggaaaagtctcacaagcttttaaccaagttgaatcacgacgacaacgataaagaaatcctcaaccatctaacacatgaagtacaaagtagaaatgtgatcttattggacaaactagaggagctcaacaaggaactgggctggattaaagaccgaaaatgaggaaccatgagcactgggcgtttccagaaaaactgcaaccaacgatgggaaaatgataccacactactatggtcaccccacattgtgaaatttcaaaccaaaaaagatcaaccccataattccccagagggttttcccaacaattttccaacggacttgataatgagtcagatcatttgagcatattcatcttaccccttattccgtgacaatttacctattccattcaaagcatacggtatcccgtgaccttctcatggagatcattctccaccgatacagcatatacacagatatacccaactaatatcaattggaccttgatatggtcgaccttgatggtcccgtccaaccttaaaacttagtttaatgctatactttcgccttgaaccaaatctgtctccccctcaatcatctctatgcaagaaggtcaacactgattacgtgagcaacagccagcaatcgttcgagtccccgccaaaaaaggcggagttactgctccttgtgaccacaccccctgagaccacgtccctaaacgatccttgtcggttccttcgtccaattggcaattgccacgcatacgtgaatcgttattgtttcgcctaccttgcgtcattcgttccagaatgttcgacatactcctctagaacataccgtcacaccaccatcttaagttatcttcacgtgaccatgacgtacattgtagttgactaccccattctcatcattccgatgcggccaaaaatctctatataaagaccgtatcccctaatattctcttcttgttaagacattaacttagttaattcaccaattactcacttataaacaaacaaa

SEQ ID NO: 7 - DNA, 1500 bp, synthetic construct
  misc_feature 1..1500: note = Description of Artificial Sequence: Synthetic Oligonucleotide
  source 1..1500: mol_type = other DNA
gtttctcttggggagatacttttttcgcgtgctcctccgtgcggaacttccttctgagcttctacctctcagattagtctaatcgcatcaggaataagactgagaatgcttttaaggagaggcttgagattggctaattgcgttccgaagtactctttcaaaaggagttatacccctctcaactacgattctctaaagaattatcgtaggcatgctcaggcgcctcaaccccatcagtttgacgccactagatgggaccaacaaccagttactaatgagcaaggagtaatactcccatccgactcaattgcaaacattctgagacaaccaactctggtcatagaacggcaaatggaaatgatgaatatatttttaggatttgagcaggcgaaccgatatgttatcatggatcctacaggaagtattttgggttacatgctagaaagggatctgggcatcaccaaagctatattgagacagatctaccgtttgcatcgaccttttacagtggatgtaatggatactgcaggaaatgtattaatgacaatcaagaggccgtttagtttcatcaattcgcacatcaaagctatattaccccctttcaggaacagcgacccagacgaacatgtaattggagaatccgttcaaagctggcatccttggagacgaagatacaatctatttacagcacaaattggcgaaaaggacactgtctacgatcagttcgggtacattgacgcaccgtttctttcctttgagtttcctgtactttcagaatctaggcaaacgctaggtgctgtctctagaaacttcgtgggctttgcaagagagcttttcacagatacaggagtttacatcatccgtatggggcctgaatcttttgtagggctagaagggaactacgggaacaatgtggcccaacatgcccttacgctggaccaaagggctgtattattagccaatgccgtttcaattgactttgattacttttctaggcactcgtcacacagtggtggcttcattgggtttgaggaatagacagggtctcgtcaactcagctcctgccaccaaaccaatcattgatcaacgagcacacttttgtccacgtgagatcgctttcgcttgcagaaagagcaatgcatgaaaacggcaaacgcaaaacgagcaaaaaaacgagtaaataactacaatttcaccaccaacagggtcaaagagcttttgagacactataaaaggggccctttccccccaggttccttgaaatcctcattcaattatgttttttactcataatttgactcaattggcatcttcttctttgttcatatacagtaattgatatgacgcttagtcattattagtgttctcgactagcagtggcgaaaaaagggggagttattttctagaaccgaccgcaaactataaaagaaagctgcccctcatatacctttcgaattctttattttctgtgtttcttccctatttaacatctacacaaaa